(19) United States
(12) Patent Application Publication
(10) Pub. No.: US 2017/0183676 A1
(43) Pub. Date: Jun. 29, 2017

(54) ENHANCING BIOCHEMICAL OUTPUT TRAITS IN CROPS

(71) Applicant: PURDUE RESEARCH FOUNDATION, West Lafayette, IN (US)

(72) Inventors: Clint C. S. Chapple, West Lafayette, IN (US); Natalia Dudareva, Lafayette, IN (US); Nicholas Bonawitz, Lebanon, IN (US); Jeong Im Kim, West Lafayette, IN (US)

(73) Assignee: PURDUE RESEARCH FOUNDATION, West Lafayette, IN (US)

(21) Appl. No.: 15/223,874

(22) Filed: Jul. 29, 2016

Related U.S. Application Data
(60) Provisional application No. 62/200,878, filed on Aug. 4, 2015.

(51) Int. Cl. C12N 15/82

(52) U.S. Cl. CPC C12N 15/8243; C12N 15/8218

(57) ABSTRACT

Presented herein are methods to enhance phenylpropanoid metabolite production.

### CROSS-REFERENCE TO RELATED APPLICATIONS

The present U.S. patent application is related to and claims the priority benefit of U.S. Provisional Patent Application Ser. No. 62/200,878, filed Aug. 4, 2015, the contents of which is hereby incorporated by reference in its entirety into this disclosure.

### STATEMENT OF GOVERNMENT INTEREST

This invention was made with government support under DE-FG02-07ER15905 awarded by the Department of Energy. The government has certain rights in the invention.

### TECHNICAL FIELD

The present disclosure generally relates to increasing plant quality and yield, and in particular to a method for manipulating of phenylpropanoid metabolism and output trait improvement in agriculturally and horticulturally important plants.

### BACKGROUND

This section introduces aspects that may help facilitate a better understanding of the disclosure. Accordingly, these statements are to be read in this light and are not to be understood as admissions about what is or is not prior art.

As sessile organisms, plants produce a variety of secondary metabolites that are thought to number in the hundreds of thousands. Many of these are derived from the amino acid phenylalanine and are known as phenylpropanoids, a class of compounds that includes health-promoting phytonutrients. Consumption of plant foods rich in phytonutrients is more beneficial to human health than the use of isolated phytochemicals as food supplements. To increase the intake of nutritional foods to meet dietary requirements, the overall palatability of fruits and vegetables, which is determined by their aroma and flavor, has to be improved along with the nutritional quality. Tomato is the second most commonly grown vegetable crop in the world after potato. In the American diet, tomatoes are the fourth most commonly consumed fresh vegetable (˜18 pounds per capita/year) and the most frequently consumed processed product (˜69 pounds per capita/year). Wide consumption of tomatoes around the world makes it one of the principle sources of phytonutrients and subsequently one of the preferred targets for metabolic engineering. Tomato phenylpropanoids also include volatile compounds such as methylsalicylate, phenylacetaldehyde and phenylethanol, the latter of which substantially contribute to tomato flavor and serve as positive indicators of the nutritional value of the fruits.

Another group of important plant phenylpropanoid metabolites are floral scent compounds that play a key role in attracting and guiding pollinators. Many crop plants depend on pollinators for successful reproduction over multiple generations. In fact, about one-third of leading food plants including berries, nuts, oilseeds, fruit trees and vegetables are directly or indirectly dependent on pollinators for their reproduction. Unfortunately, the rapid decline of the population of honeybees has raised concerns because of the potential for direct impact on plant fertility. One strategy to make up for the declining bee population is to increase the attractiveness of flowers for the limited pool of pollinators. Although various floral characteristics such as pigmentation and nutritional value are important to pollinators, floral scent is essential to attract pollinators from a distance. It is anticipated that enhancement of floral scent production will more efficiently attract pollinators, which in turn will have a beneficial impact on agricultural crop productivity. Unfortunately, manipulation of the production of these molecules is very challenging because of the complex nature of the biosynthetic pathways involved and their tight regulation. The most important limitation to the manipulation of scent production is that in most plants, many of genes involved in the biosynthetic pathways leading to these emitted compounds have not yet been identified. Therefore, there is an unmet need for novel approaches to up-regulate floral scent production that do not require identification of dozens of target genes that may need to be overexpressed individually in order to increase floral scent.

### SUMMARY

In one aspect, a method is presented to enhance phenylpropanoid metabolite production, which includes regulating production of phenylpropanoid molecules in a plant to thereby enhance nutritional value. In another aspect, a method is presented to enhance phenylpropanoid metabolite production, which includes regulating production of phenylpropanoid molecules in a plant to thereby enhance organoleptic qualities. In yet another aspect, a method is presented to enhance phenylpropanoid metabolite production, which includes regulating production of phenylpropanoid molecules in a plant to thereby enhance floral scent production.

In an embodiment, the methods can further include mutating a plurality of genes, the genes encode MED5 subunits. In another embodiment, the methods can further include down-regulating the plurality of genes. In yet another embodiment, the methods can further include down-regulating the plurality of genes. In yet another embodiment, the plant is a crop. In yet another embodiment, the plant is a flower.

### BRIEF DESCRIPTION OF THE FIGURES

FIG. 1 is a Bayesian phylogenetic tree of plant MED5 homologs.

FIG. 2 shows conserved amino acid residues of MED5 in Arabidopsis, tomato (S. lycopersicum) and P. hybrida.

FIG. 3a shows expression profiles of putative MED5 candidates in P. hybrida. Transcript levels were determined by qRT-PCR and shown relative to the reference gene (ubiquitin). Data are means±SE (n=3 biological replicates). Tissue-specific, rhythmic and developmental expression profiles for Comp16558 are shown. Tissue-specific expression of the candidates is shown relative to the corresponding transcript levels in corolla tissue. Rhythmic expression of candidates from day 1 to day 3 during a normal light/dark cycle in corolla tissue. Developmental changes in expression of the candidates from mature buds to day 7 post-anthesis. Transcript levels are shown relative to the day/time of highest expression.

FIG. 3b shows expression profiles of putative MED5 candidates in P. hybrida. Transcript levels were determined by qRT-PCR and shown relative to the reference gene (ubiquitin). Data are means±SE (n=3 biological replicates). Tissue-specific, rhythmic and developmental expression profiles for Comp20239. Tissue-specific expression of the candidates is shown relative to the corresponding transcript levels in corolla tissue. Rhythmic expression of candidates from day 1 to day 3 during a normal light/dark cycle in corolla tissue. Developmental changes in expression of the candidates from mature buds to day 7 post-anthesis. Transcript levels are shown relative to the day/time of highest expression.

FIG. 3c shows expression profiles of putative MED5 candidates in P. hybrida. Transcript levels were determined by qRT-PCR and shown relative to the reference gene (ubiquitin). Data are means±SE (n=3 biological replicates). Tissue-specific, rhythmic and developmental expression profiles for Comp21779. Tissue-specific expression of the candidates is shown relative to the corresponding transcript levels in corolla tissue. Rhythmic expression of candidates from day 1 to day 3 during a normal light/dark cycle in corolla tissue. Developmental changes in expression of the candidates from mature buds to day 7 post-anthesis. Transcript levels are shown relative to the day/time of highest expression.

FIG. 4a shows the effect of transient overexpression of AtREF4-3 on emission of benzenoid/phenylpropanoid volatiles in corollas of P hybrida flowers. AtREF4-3 transcript levels in empty vector control (white bar) and infiltrated flowers (black bar) determined by qRT-PCR (means±SE n=4 biological replicates). All emission levels in infiltrated flowers are expressed relative to emission levels in the empty vector control set at 100%. Scent collections were performed on detached infiltrated flowers from 4 PM to 11 PM. Data are means±SE (n=4 biological replicates). *P<0.1 and **P<0.05 by student's t test relative to control.

FIG. 4b shows the effect of transient overexpression of AtREF4-3 on emission of benzenoid/phenylpropanoid volatiles in corollas of P hybrida flowers. Levels of benzenoid/phenylpropanoid volatiles in empty vector control (white bars) and infiltrated flowers (black bars). All emission levels in infiltrated flowers are expressed relative to emission levels in the empty vector control set at 100%. Scent collections were performed on detached infiltrated flowers from 4 PM to 11 PM. Data are means±SE (n=4 biological replicates). *P<0.1 and **P<0.05 by student's t test relative to control.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In response to the unmet need for novel approaches to up-regulate floral scent production that do not require identification of dozens of target genes that may need to be overexpressed individually in order to increase floral scent, a method for manipulation of phenylpropanoid metabolism and output trait improvement in agriculturally and horticulturally important plants is presented herein.

Enhancing plant yield and the nutritional value of food has taken on an urgent renewed importance in the face of an increasing global population and escalating demands for food. Plant metabolites derived from the amino acid phenylalanine (phenylpropanoids) have an inestimable impact on plant and human health. For humans, many of these compounds are important flavor and nutritional components, particularly those that serve as anti-oxidants. Floral scent molecules are critically important to plants that rely on pollinators such as bees for their successful reproduction. Unfortunately, a decline in the population of pollinators has raised concerns because of its potential impact on agriculture and the food supply. Finally, one of the most enjoyable traits of cut flowers is their smell and yet in the course of their domestication, floral scent production has been bred out of many varieties. Here we propose an entirely new molecular approach to enhance the production of plant metabolites vital to plant reproduction, organoleptic properties and human nutrition. We have shown that the transcriptional co-regulator Mediator suppresses phenylalanine-derived compound (phenylpropanoid) production in Arabidopsis. The absence of Arabidopsis Mediator subunit 5A (AtMED5A) and its paralog, AtMED5B results in the over-expression of an array of genes required for phenylpropanoid biosynthesis and in turn enhanced phenylpropanoid accumulation. We identify MED5 homologs in Petunia hybrida, and down-regulate their expression to enhance floral scent production. These experiments serve as a model for the manipulation of phenylpropanoid metabolism and output trait improvement in other agriculturally and horticulturally important plants.

As a model plant, we use Petunia hybrida cv Mitchell because well-proven genetic engineering tools are available, RNAseq databases have been developed, and its floral scent biosynthetic pathways have been well established. While Petunia hybrid cy Mitchell has been utilized as a model plant in the present disclosure, such use is for demonstrative purposes only and is not intended to be limiting on the scope of the invention.

Arabidopsis Mediator subunits, MED5A and MED5B, regulate the production of phenylpropanoid compound in the model plant Arabidopsis. Mediator is a multi-protein complex that serves as a transcriptional co-regulator for basal and regulated transcriptional events in all eukaryotes. Some subunits are required for global transcription whereas others are required for transcriptional regulation of very specific processes. When we mutate the genes encoding MED5A and MED5B in Arabidopsis, a suite of genes involved in phenylpropanoid compound biosynthesis are upregulated and the level of phenylalanine-derived compounds increases without significant alteration of growth and development. These data demonstrate that MED5A/B functions as a master regulator of phenylpropanoid compound accumulation that can be used to manipulate this important pathway in agriculturally important plants. While Arabidopsis does not produce phenylpropanoid volatiles, many plants including petunias produce a variety of them including methylbenzoate, benzaldehyde, benzylalcohol, phenylacetaldehyde, eugenol, isoeugenol, benzylbenzoate and phenylethylbenzoate. Because Mediator is conserved throughout eukaryotes, it can be anticipated that repression of the expression of MED5 homolog(s) in petunia or other flowers, and ultimately other crops important for human nutrition will increase production of important or valuable phenylpropanoid compounds. We test this hypothesis using petunia, a plant that produces high levels of Phe-derived VOCs and is one of the top 5 annual bedding plants in the United States with a total wholesale value of $130 million in 2012. Although VOC biosynthesis in petunias has been extensively studied, MED5 homologs in petunias and its related plants have not yet been identified nor manipulated. In parallel, we take similar approaches to manipulate the production of the important anti-oxidants and flavor components found in tomato. Methods for the down regulation of MED5 homologs in plants include antisense and co-suppression approaches, RNA interference methods, or targeted gene knockouts using technologies such as zinc finger nucleases and Crispr/Cas9. Similar to petunia, it can be anticipated that repression of the expression of MED5 homolog(s) in tomato fruits can increase production of important or valuable phenylpropanoid compounds.

### EXAMPLE

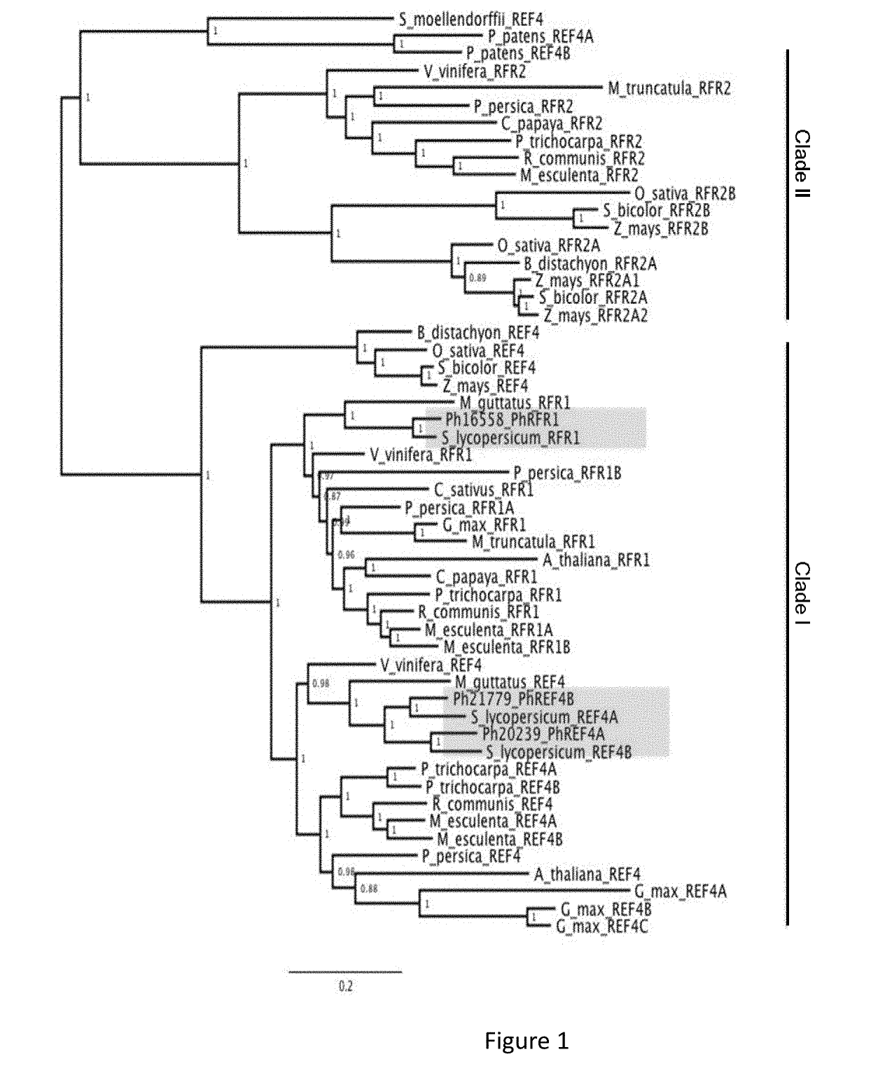

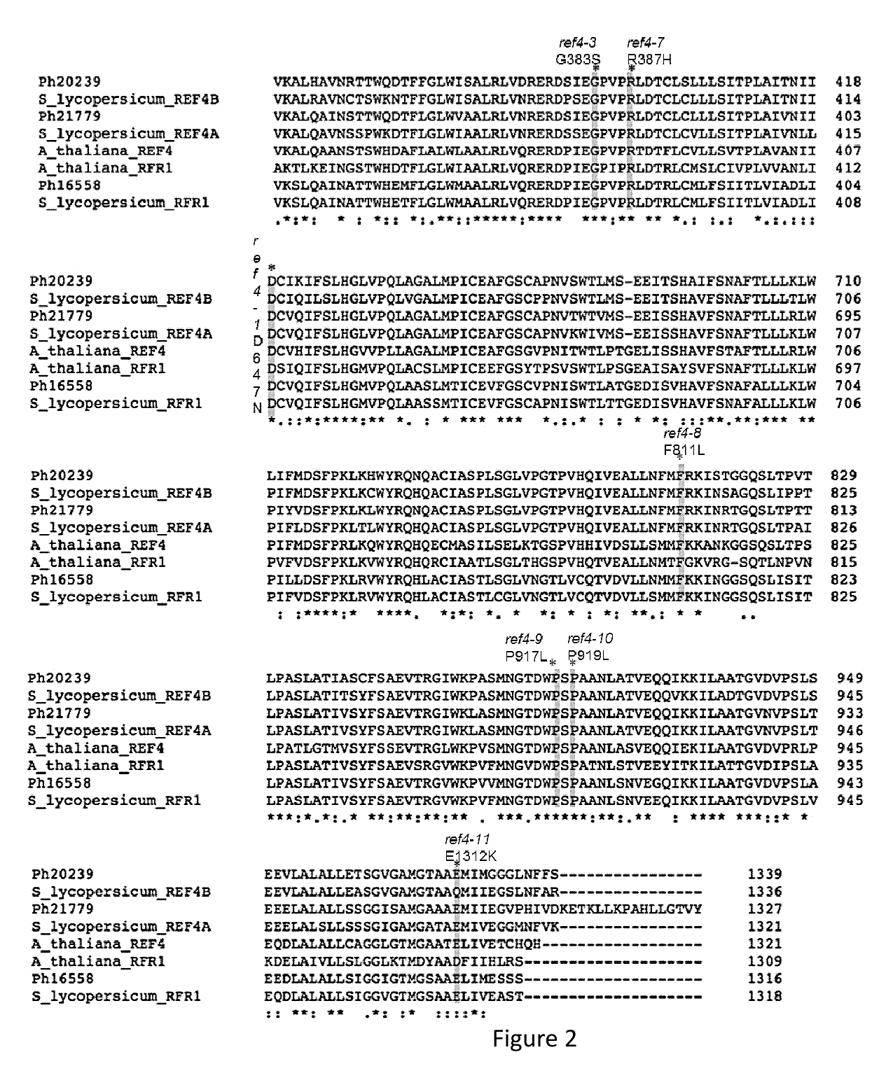

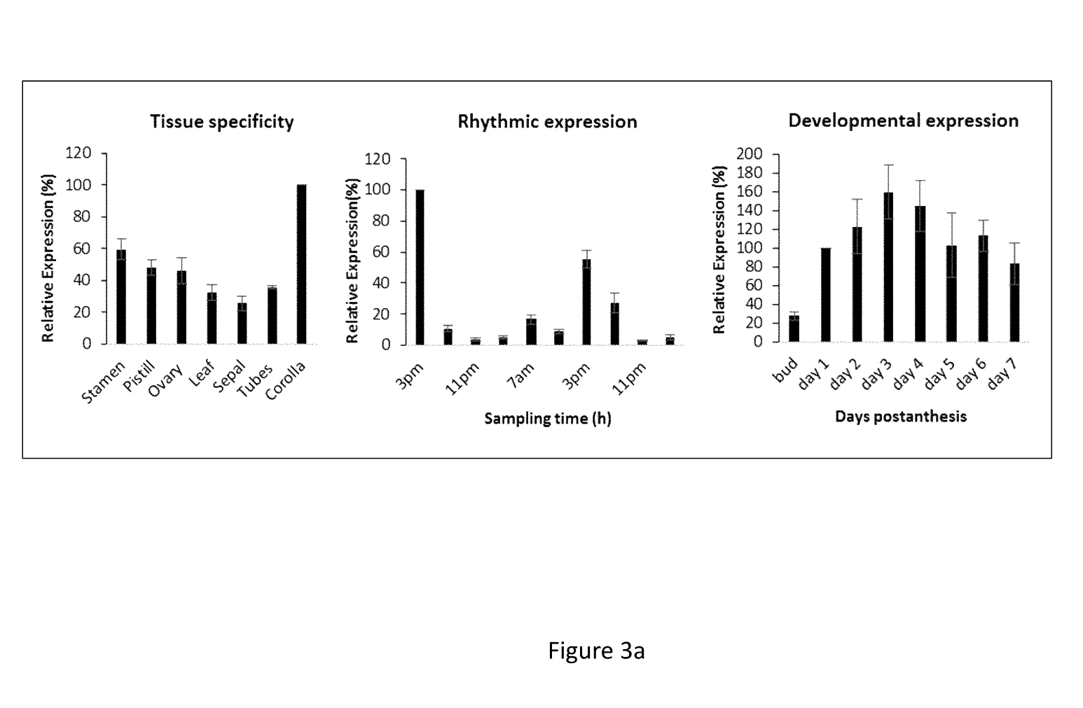

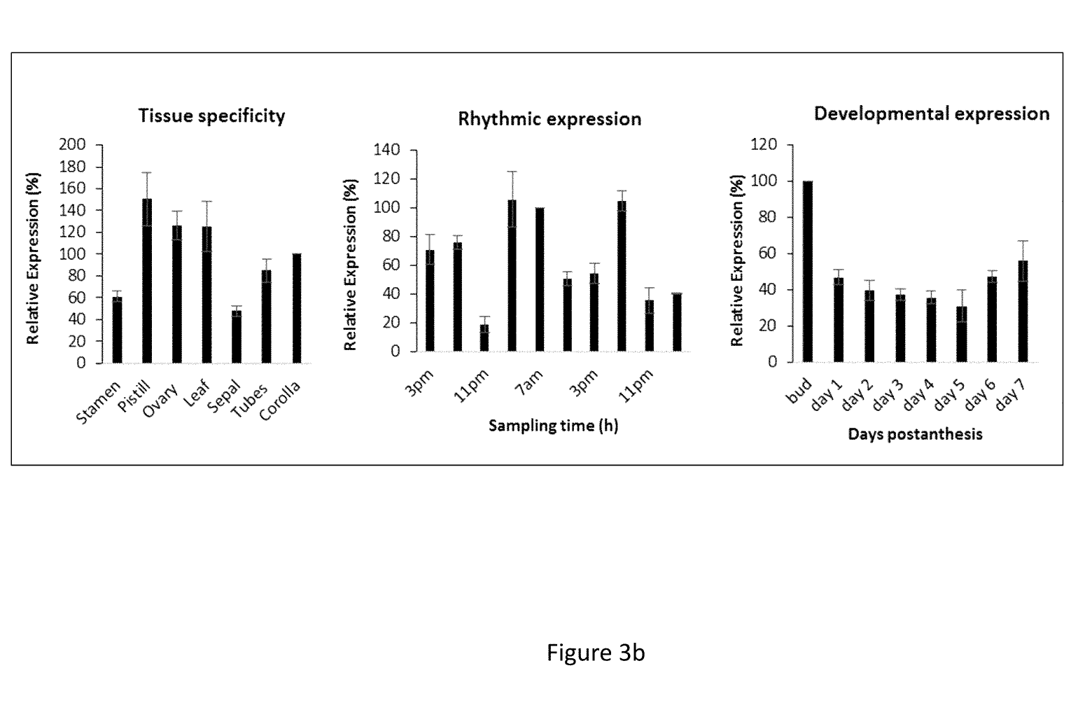

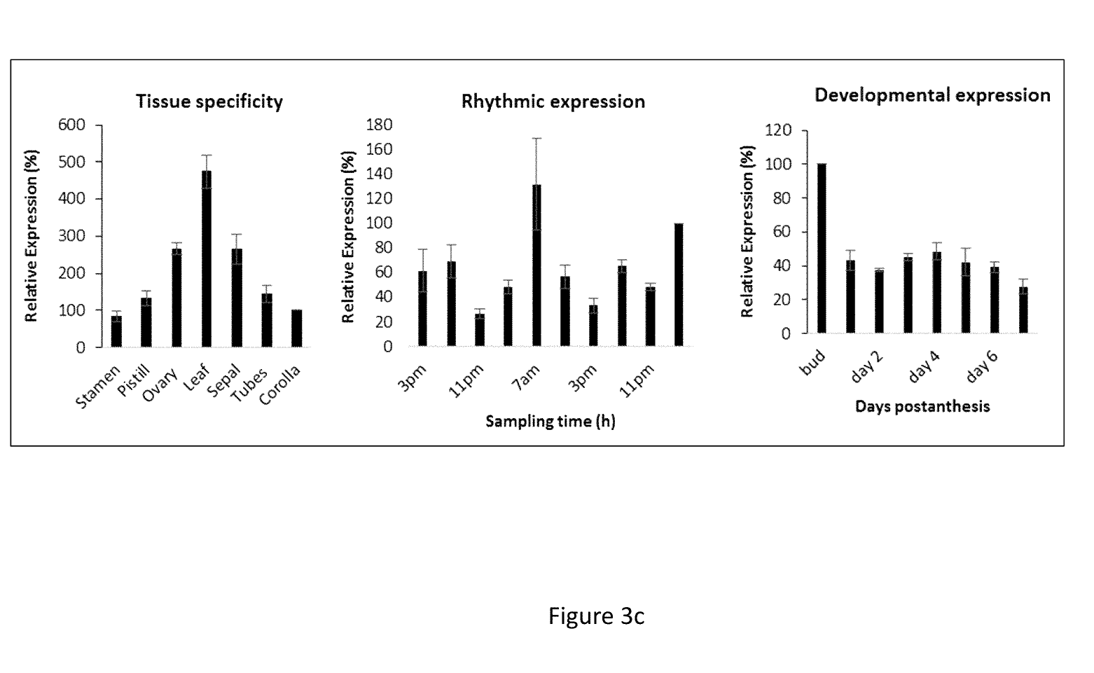

We have found that P. hybrida has 3 MED5 paralogs (Table 1), all of which are Clade I members (FIG. 1). No Clade II members, commonly found in dicotyledonous plants, were identified. Three MED5 members were similarly identified from tomato, all of which share a high degree of identity with those from P. hybrida as well as key amino acid residues with more distantly related plant MED5 subunits (FIGS. 2 and 3a-3c). With this sequence information, quantitative real time PCR primers were designed to examine the expression of each P. hybrida MED5 paralog. These experiments revealed that one paralog (Comp16558) is expressed most strongly in the corolla, another (Comp21779) in leaves and that the third (Comp20239) exhibits little organ specificity (FIGS. 3a-3c). None of the genes exhibited convincing rhythmic expression although the paralog expressed most highly in corolla exhibited a developmental profile similar to scent-related enzyme genes.

TABLE 1
Three petunia MED5 homologs and three tomato MED5 homologs were
identified from petunia RNAseq and genomic sequence databases, and Genbank.

| | | | similarity | | similarity |
|---|---|---|---|---|---|
| | amino | identity with | with | identity with | with |
| | acid | AtREF4 | AtREF4 | AtRFR1 | AtRFR1 |
| | length | (%) | (%) | (%) | (%) |
| Ph20239/PhREF4A | 1339 | 58.7 | 73.3 | 56.7 | 72.6 |
| Ph21779/PhREF4B | 1327 | 61.9 | 75.4 | 58.1 | 75.1 |
| Ph16558/PhRFR1 | 1316 | 60.2 | 75.0 | 63.4 | 78.3 |
| S_lycopersicum_REF4A | 1340 | 60.7 | 74.8 | 57.6 | 74.7 |
| S_lycopersicum_REF4B | 1348 | 59.5 | 74.0 | 55.9 | 71.9 |
| S_lycopersicum_RFR1 | 1337 | 61.0 | 75.3 | 63.2 | 78.7 |

In addition, RNAi constructs were designed for each candidate. For Comp16558, DNA containing two spliced cDNA fragments of the coding region corresponding to nucleotides 1559-2001 and 1559-1858 was synthesized (Genscript, Piscataway, N.J.) in a PENTR/SD/D-TOPO® vector for entry into a Gateway® vector (Pb2gw7). To create a hairpin structure, the 1559-1858 fragment was synthesized in an antisense orientation. Comp20239 and Comp21779 were also synthesized in a similar manner. For Comp20239, fragments of the coding region corresponding to nucleotides 2687-3130 and 2687-2978 (antisense) was synthesized. For Comp21779, nucleotides 3431-3875 and 3431-3722 (antisense) was synthesized. Once completed, these constructs can be used for transient and stable transformation of P. hybrida.

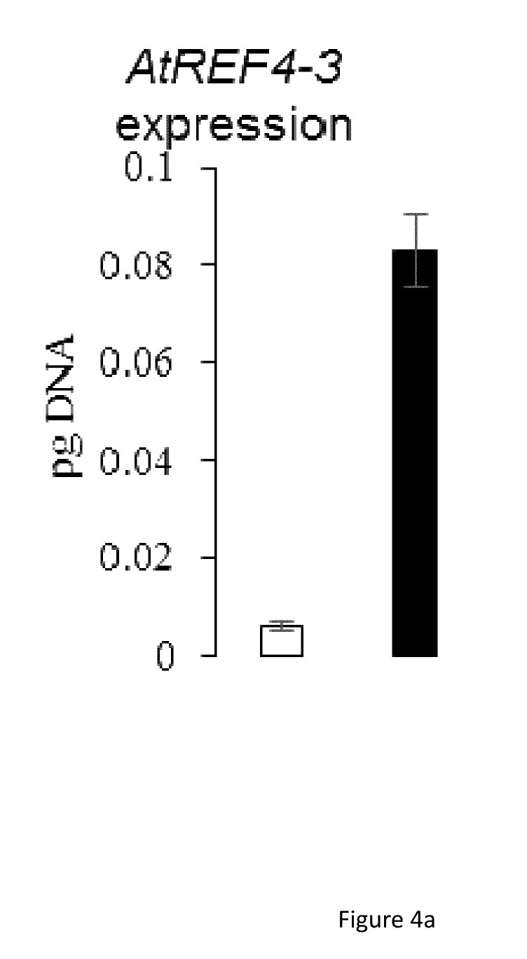

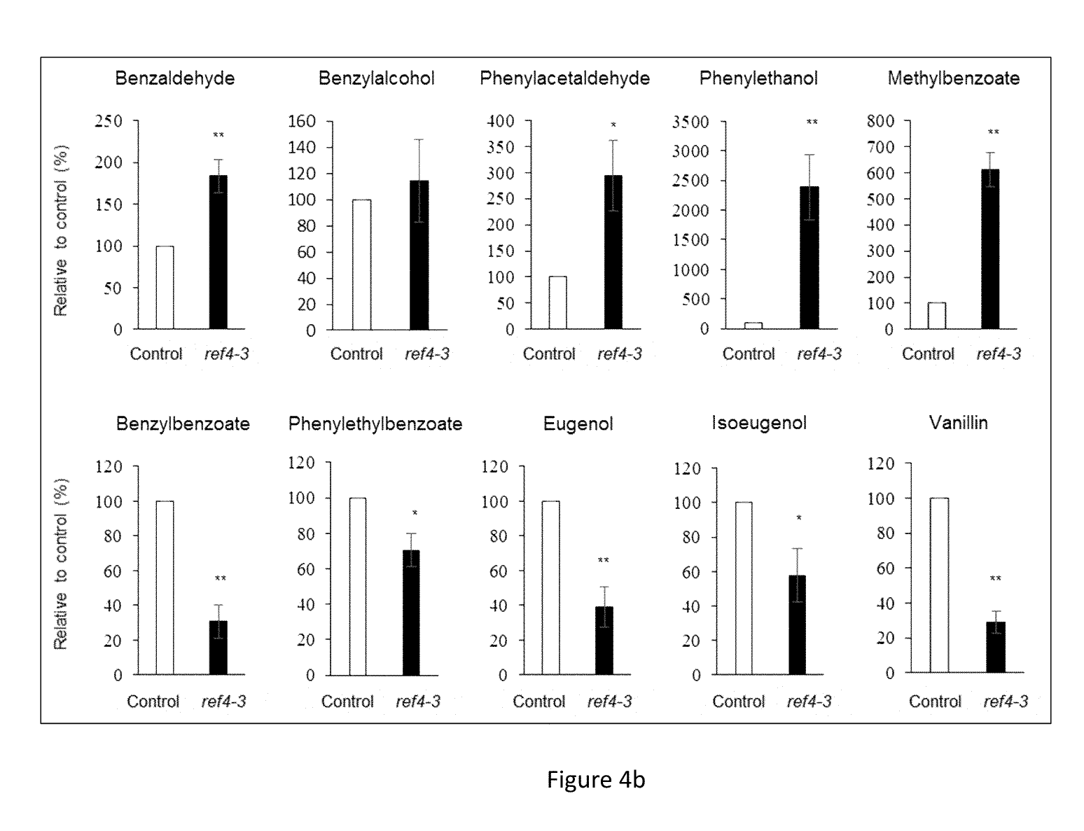

We next evaluated whether a construct we had in hand that carried a dominant negative form of the Arabidopsis MED5a gene would have an impact on P. hybrida floral volatile production when transiently transformed into flowers. Quantitative real time PCR analysis demonstrated that we obtained expression of the Arabidopsis gene (FIG. 2), and floral volatile analysis by GC-MS revealed striking changes (FIGS. 3a-3c). Levels of benzylbenzoate, phenethylbenzoate, eugenol, isoeugenol, and vanillin went down; whereas levels of benzaldehyde, phenylacetaldehyde, phenylethanol, and methylbenzoate emission increased. Although the decreased emission of the first set of compounds was expected, the increases seen in the second group of compounds is both unprecedented and of great interest because it suggests an unexpected complexity of differential gene regulation caused by the dominant MED5a gene, and possible redirection of phenylalanine metabolism from phenylpropanoid derivatives to phenylethanol, an important scent molecule. It is interesting to note that the enhanced floral scent emissions (phenylacetaldehyde, phenylethanol and methylbenzoate) from transiently transformed P. hybrida flowers can be detected not only by gas chromatography as shown in FIG. 4, but by the human nose.

Those skilled in the art will recognize that numerous modifications can be made to the specific implementations described above. The implementations should not be limited to the particular limitations described. Other implementations may be possible.

### REFERENCES

1. Canene-Adams K, Campbell J K, Zaripheh S, Feffery E H, and Erdman Jr. J W. The tomato as a functional food. J. Nutr. 135:1226-1230 (2005)
2. Goff S A and Klee H J. Plant volatile compounds: Sensory cues for health and nutritional value? Science 311:815-819 (2006)
3. Rodriguez-Saona C R, Rodriguez-Saona L E, and Frost C J. Herbivore-induced volatiles in the perennial shrub, Vaccinium corymbosum, and their role in inter-branch signaling. J. Chem. Ecol. 35:163-175 (2009)
4. Raguso R A. Wake up and smell the roses: The ecology and evolution of floral scent. Annu. Rev. Ecol. Evol. Syst. 39:549-569 (2008)
5. Huang M, Sanchez-Moreiras A M, Abel C, Sohrabi R, Lee S, Gershenzon J and Tholl D. The major volatile organic compound emitted from Arabidopsis thaliana flowers, the sesquiterpene (E)-β-caryophyllene, is a defense against a bacterial pathogen. New Phytol. 193:997-1008 (2012)
6. Kevan P G, Clark E A, and Thomas V G. Insect pollinators and sustainable agriculture. Am. J. Agr. Econ. 5:13-22 (1990)
7. vanEngelsdorp D, Caron D, Hayes J, Underwood R, Henson M, . . . and Pettis J. A national survey of managed honey bee 2010-11 winter colony losses in the USA: results from the Bee informed partnership. J. Apic. Res. 51:115-124 (2012).
8. Bonawitz N D, Soltau W L, Blatchley M R, Powers B L, Hurlock A K, Seals L A, Weng J K, Stout J, and Chapple C. REF4 and RFR1, subunits of the transcriptional coregulatory complex Mediator, are required for phenylpropanoid homeostasis in Arabidopsis. J. Biol. Chem. 287:5434-5445 (2012)
9. Bonawitz N D, Kim J I, Tobimatsu Y, Ciesielski P N, Anderson N A, . . . and Chapple C. Disruption of Mediator rescues the stunted growth of a lignin-deficient Arabidopsis mutant. Nature 509:376-380 (2014)
10. Poss Z C, Ebmeier C C, and Taatjes D J. The Mediator complex and transcription regulation. Crit. Rev. Biochem. Mol. Biol. 48:575-608 (2013)
11. Dudareva N, Klempien A, Muhlemann J K, and Kaplan I. Biosynthesis, function and metabolic engineering of plant volatile organic compounds. New Phytol. 198:16-32 (2013)
12. USDA, National Agricultural Statistics Service. Floriculture crops 2012 summary. http://usda01 library.cornelLedu/usda/current/FlorCrop/FlorCrop-04-25-2013.pdf
13. Cseke L, Dudareva N and Pichersky E. Structure and evolution of linalool synthase. Mol. Biol. Evol. 15:1491-1498 (1998)
14. Orlova I, Marshall-Colon A, Schnepp J, Wood B, Varbanova M, . . . and Dudareva N. Reduction of Benzenoid synthesis in Petunia flowers reveals multiple pathways to benzoic acid and enhancement in auxin transport. Plant Cell 18:3458-3475 (2006)
15. Yoo H, Widhalm J R, Qian Y, Maeda H, Cooper B R, Jannasch A S, Gonda I, Lewinsohn E, Rhodes D and Dudareva N. An alternative pathway contributes to phenylalanine biosynthesis in plants via a cytosolic tyrosine:phenylpyruvate aminotransferase. Nat. Commun. doi:10.1038/ncomms3833 (2013)
16. The tomato genome consortium. The tomato genome sequence provides insights into Fleshy fruit evolution. Nature 485:635-641 (2012)
17. Horsch R B, Fry J E, Hoffmann N L, Eichholtz D, Rogers S G, and Fraley R T. A simple and general method for transferring genes into plants. Science 227:1229-1231 (1985)
18. US Bureau of Economic Analysis; Personal Consumption Expenditures by type of product (2013)

1. A method to enhance phenylpropanoid metabolite production, comprising regulating production of phenylpropanoid molecules in a plant to thereby enhance nutritional value.

2. A method to enhance phenylpropanoid metabolite production, comprising regulating production of phenylpropanoid molecules in a plant to thereby enhance organoleptic qualities.

3. A method to enhance phenylpropanoid metabolite production, comprising regulating production of phenylpropanoid molecules in a plant to thereby enhance floral scent production.

4. The method of claim 1, further comprising mutating a plurality of genes, the genes encode MED5 subunits.

5. The method of claim 1, further comprising down-regulating the plurality of genes.

6. The method of claim 1, wherein the plant is a crop.

7. The method of claim 1, wherein the plant is a flower.


SEQUENCE LISTING (10 sequences)

SEQ ID NO: 1 - DNA, 3828 bp, Petunia x hybrida
atggctccgtcggagttccaaccgagtctctgggaatcggttacgtcgctgattaggtcagcccaggagaagaacgtggatccgcttcattgggctcttcagcttcgcttgaccctcgcgtctgctggaatctcccttccctctccggacctcgctcaattcctcgtcacccacatcttctgggaaaatcactctcctctttcatggaagcttctcgaaaaagctatatccgtcaatattgtccctcccctacttgtcctcgcccttctctccccaagggtaatcccaaacagaaagcttcatcccgcagcctataggctctatatggaactcctcaagagacatgcattctcattcatgcctctaatcagagcaccaggatatcacaaaacaatgaattccatcgatgatatcctccatctttctgaaacctttggtgtccaagatcaagaaccgggaagtattctccttgcatttgtcttctccattgtctgggagctacttgatgcatccctagacgaggaagggcttttagaactgacatctaataagagatctaaatggccaagcagcccacacgacatggatttagacgggctggaaaattctgtgaagagaaatgaaaaccatgatgcactagagaaagcaaacactgaaatggctatcgagctaatccaagaatttttgcagaataaagtcacatcgaggattcttcatttggcaagtcaaaacatggagagcaagaccatccctcgaggagaatttcatgcgattgtctcctctggttctaagttagcattgaccagtgattctgcactttggttgcccattgatcttttcttcgaagacataatggatgggacacaagctgcagcagctagcgctgttgaaaatctaaccggtcttgtaaaagcattgcaagcagctaatagtacatcatggcatgatgcctttctagctctatggttagcagccttacgccttgtgcaaagggagaatctatgtctgcgttattgctttttcatgcatatgttggaaatattatctgaagaaagagatccaatcgagggtcctgtgccccgcaccgacacttttctttgtgtattactttctgtcactccacttgcagttgccaatattatcgaagaagaagaaagtcagtggattgatcaaacttcgtctagtccaagcaaccagtggaaagagaaaaaaggaaaatgtcgacaaggattaattaatagcttacaacaacttggtgattatgaaagcttgttgacaccacctcgttctgtacagtccgttgccaatcaagctgctgctaaagcaattatgttcatctccggcattacaaatagtaatggatcctatgaaaatacaagcatgagcgaatctgcaagtggctgctgtaaagtgcggttttccttgtttactctaaagatgtttgttgttatgggtgtgtatctgctctgtaacatttcttgttggtctctagtgatgaagggttctcctctaacaccgtcattgacaaattcactaatcacaactccagcttctagcttagcagagattgaaaagatgtacgaggttgcaactactggttcagaagatgagaagatagccgtggcttctattctctgtggagcatctctgtttcgtggttggagcatacaggagcatgttattatcttcattgtaactttactttcgcctccggctccagctgatctttctgggagttatagccatctaatcaattctgcgccgtttcttaatgttcttcttgtcgggatatcacccatagattgtgttcacattttctcattgcacggggtggtcccattacttgctggtgctttgatgccaatatgtgaagcgtttggctccggcgttccaaacattacgtggactctcccgactggcgaattaatctcctctcatgctgttttctccactgcatttacacttcttctgaggctatggagatttgatcacccaccactagattacgtcttgggagatgttcccccggtgggccctcaacccagccctgagtatctgttgttagtaagaaattgccgtctggaatgttttggaaagtccccaaaggatcgcatggcacgtcgaagattttcgaaagtgatagatatctctgtggatcccatcttcatggattcattccccagactgaaacagtggtaccggcagcatcaggaatgtatggcttcaattctctctgaactaaagacaggaagcccagtgcatcacattgtcgattccctccttagcatgatgttcaagaaggcaaacaaaggtggtagtcagtcactgaccccatcttcagggagcagtagtttatctacttctggaggtgatgactcgtctgatcaactcaagttacctgcatgggatatcttggaagcggcmccgtttgtgcttgatgctgctctaactgcttgtgctcatggatcactctctccccgggaactagcaacaggtcttaaaatcttggctgattttctgccggcaacacttggaaccatggttagctacttttcttcggaagtaacacgaggtctgtggaagcccgtttccatgaacggaactgactggcctagtcctgctgcaaatttagcaagcgttgagcaacagatagagaagatcttagcagctactggcgttgacgtaccaaggcttccagcagatggaatctctgcggctacacttcccttgcctctcgctgcactagtgagccttactataacctacaagcttgacaaagcaacagagcgcttccttgtacttgtaggaccggccttagactcccttgctgctgcttgcccctggccctgcatgcctattgtgacatcgctgtggacccagaaagtgaagcgctggagcgactttcttatcttttcagcctccagaactgtgttccaccacaacagagatgctgttattcagctcctcagaagctgcttcacttgcacccttggtctaacacctacttcccaactctgcagctatgggggagtgggtgcactccttggccatggctttgggtcccgttactctggaggaatttccacagctgccccaggaatcttgtacataaaagtgcacagatcgatcagagatgtcatgttcctgacagaagaaatcctgtctcttctgatgttctctgtgaagagcattgccacgagggagctaccagcagggcaagcagagaaattgaagaaaacgaaagatgggtcgaggtacgggattgggcaggtttcactttctttagcaatgagacgtgtcaagctggcagcttccctgggagcctctctggtgtggatctccggtggcctaaacctggttcaggcactgatcaaggagacattaccgtcttggttcatctcggttcacggagaagaagatgagttgggaggtatggttccgatgctaagaggctatgctttagcctactttgcaatcctgagcagtgcatttgcctggggagtggactcctcatatcctgcatcaaaacgtcggccaagggttctgtggctgcatttggagtttatggtgagtgccttggaagggaagatatcactgggctgtgattgggcgacatggcaagcttacgtgacaggctttgtgagcctgatggtgcaatgcacgccggcttgggtgctagaggtggatgttgaagtaataaagagactaagcaaaagtctgaggcagtggaacgagcaagacttggctctggctctgctttgtgccggcggccttgggacaatgggtgcagctacagaactcattgtggagacttgccatcaacattag

SEQ ID NO: 2 - protein, 1275 aa, Petunia x hybrida
MAPSEFQPSLWESVTSLIRSAQEKNVDPLHWALQLRLTLASAGISLPSPDLAQFLVTHIFWENHSPLSWKLLEKAISVNIVPPLLVLALLSPRVIPNRKLHPAAYRLYMELLKRHAFSFMPLIRAPGYHKTMNSIDDILHLSETFGVQDQEPGSILLAFVFSIVWELLDASLDEEGLLELTSNKRSKWPSSPHDMDLDGLENSVKRNENHDALEKANTEMAIELIQEFLQNKVTSRILHLASQNMESKTIPRGEFHAIVSSGSKLALTSDSALWLPIDLFFEDIMDGTQAAAASAVENLTGLVKALQAANSTSWHDAFLALWLAALRLVQRENLCLRYCFFMHMLEILSEERDPIEGPVPRTDTFLCVLLSVTPLAVANIIEEEESQWIDQTSSSPSNQWKEKKGKCRQGLINSLQQLGDYESLLTPPRSVQSVANQAAAKAIMFISGITNSNGSYENTSMSESASGCCKVRFSLFTLKMFVVMGVYLLCNISCWSLVMKGSPLTPSLTNSLITTPASSLAEIEKMYEVATTGSEDEKIAVASILCGASLFRGWSIQEHVIIFIVTLLSPPAPADLSGSYSHLINSAPFLNVLLVGISPIDCVHIFSLHGVVPLLAGALMPICEAFGSGVPNITWTLPTGELISSHAVFSTAFTLLLRLWRFDHPPLDYVLGDVPPVGPQPSPEYLLLVRNCRLECFGKSPKDRMARRRFSKVIDISVDPIFMDSFPRLKQWYRQHQECMASILSELKTGSPVHHIVDSLLSMMFKKANKGGSQSLTPSSGSSSLSTSGGDDSSDQLKLPAWDILEAAPFVLDAALTACAHGSLSPRELATGLKILADFLPATLGTMVSYFSSEVTRGLWKPVSMNGTDWPSPAANLASVEQQIEKILAATGVDVPRLPADGISAATLPLPLAALVSLTITYKLDKATERFLVLVGPALDSLAAACPWPCMPIVTSLWTQKVKRWSDFLIFSASRTVFHHNRDAVIQLLRSCFTCTLGLTPTSQLCSYGGVGALLGHGFGSRYSGGISTAAPGILYIKVHRSIRDVMFLTEEILSLLMFSVKSIATRELPAGQAEKLKKTKDGSRYGIGQVSLSLAMRRVKLAASLGASLVWISGGLNLVQALIKETLPSWFISVHGEEDELGGMVPMLRGYALAYFAILSSAFAWGVDSSYPASKRRPRVLWLHLEFMVSALEGKISLGCDWATWQAYVTGFVSLMVQCTPAWVLEVDVEVIKRLSKSLRQWNEQDLALALLCAGGLGTMGAATELIVETCHQH

SEQ ID NO: 3 - DNA, 3930 bp, Petunia x hybrida
atggtggttcctggacggagaaccgtctgggattgtgtgattgagctgacaaagatggctcaagagaactgtgtcgatcctcgtctttgggcttctcagttatcatcgaatttgaaattcttcgccgtggagttaccgtcgacggagcttgcggaggttattgtttcgtacatttgttgggataataacgttcctattgtttggaagtttcttgaaagagctatggctttgaagcttgtttctcctctcgttgttcttgctcttctcgctgacagagttgttccaacccggtctacgcaacaagcagcttatagaatttacttggagctgctcaagagaaatatgttcaccattaaagatcatattagtggacctcactaccagaaggtcatgatttcagtatccaatattcttcgtctttcagagttatttgatctggacacaagcaaacctggtgtgcttttagtagagtttgtttttaagatggtgtcacaattgctagatgcagcgttaagtgatgaaggtttgctagagctaagccaagactcgagctcccagtggctagttaagtctcaagatatggaaattgatgctcctgagagatataatgaaaagactggatcactagagaagttacagagtttgaataccatcatggctattgagttgatagcggaattcctcagaaatacagtgattgcgagattgctatacttggtatcttccaacagggcttcgaagtggcatgaatttgtccagaaagtgcagcttcttggagaaaattcatcggctttaaaacactcaaaggttctaaattctggggatctattacaattgatttcgaatagacgatttgggtattcttatgatagcaaagtaacttcagcacggaaatccaatgcaattgtggattttggatctctctcttcctatgctggcttatgccatggagcaagtctctcttcactttggcttcctcttgatctagtatttgaagatgcaatggatggatatcaagtaaacccaactagtgcgattgaaatcattaccggtctagctaaaacacttaaagaaattaatgggagcacttggcatgacactttcttggggctttggatagcagctctccgacttgttcaaagggaaagggatcctattgagggacctattcctcgactggacacaaggctgtgcatgtcattatgtattgtgcctcttgtggtcgcaaaccttattgaagaggggaaatacgaatctgtcatggaaaagctgcgagatgaccttgttacaagtttgcaggtcctcggtgatttccctggattgctggctcctcccaaatgcgttgtttctgctgccaacaaggctgccacaaaggcaatcttgtttttgtctggtggcaatgttggaaagtcatgttttgatgtcataaacatgaaggacatgcctgtcaactgctctggaaacatgcgccacctgatagtcgaggcttgtattgcaagaaacatactggatatgtcggcttattcgtggcctggctatgttaatggccgaatcaaccaaatacctcagagtctgccgaatgaagtaccctgctggtcatcatttgtgaaaggggctccactaaatgccgcaatggtgaatacattagtttcagttcctgcttcaagcttagcagagctcgagaaactatttgaggttgcagtcaaaggatcggatgacgagaagatatctgctgctacagttctttgcggggcatctctcacacggggttggaacatacaggaacacactgttgaatatcttacaagattactatccccacccgttccagcagattattccagggctgaaaatcatttgattggttatgcctgcatgttgaatgttgttattgttgggataggatctgttgacagcattcaaattttctctttacatggcatggttccgcaacttgcttgctcactaatgccaatttgtgaagagtttggatcttacaccccgagtgtatcatggactcttccttctggagaagcaatatcagcatattctgttttctcaaatgcattcactcttcttttaaagctatggagattcaatcatcctcctattgagcatggtgtgggagatgttcccacagttggctcccaactcactcctgaacatcttctttcagtgcgtaattcttatcttgtttcgtcggagatccttgatagagaccgaaacagaaagagactttcagaagttgcaagggcagcatcttgccagcccgtgtttgtggactcgttccccaagctgaaagtctggtataggcagcaccaaagatgcatagcagccacattatctggacttacacatggatctcccgttcatcagacagttgaagcacttctcaacatgacgttcggaaaggtcagaggcagtcagactttaaatccagtaaattcgggcacaagcagttcatcaggagctgcaagtgaagatagcaatattagacctgagtttcctgcttgggatatcctcaaagccgttccatatgttgtcgatgctgctttaaccgcttgtactcatggaaggctttcgcctcgtcagttagctacagggctgaaagacttagcagattttctcccggcatcattagcaacgatagtaagctatttctcagctgaggtgagtagaggtgtttggaagccagtattcatgaacggcgtggattggccaagtccagctacaaatctatccactgttgaggaatatataacgaaaattcttgcaacaacaggcgtcgatatccctagccttgctccaggagggagttctccggcaacgcttcctttacctctagccgcatttgttagtctaaccattacttacaaaattgacaaagcctcagagcggtttctcaacctggctggcccagctctggagtgcttggctgcaggctgcccttggccttgcatgcccattgtagcttcattatggacccaaaaggcgaaacgttggtttgacttccttgttttctctgcctctcgcaccgtcttccttcacaaccaagacgctgtgattcagctcctcagaaactgcttcagtgccactctgggtttgaatgctgcccccatgtcaaatgatggtggcgttggagctcttcttggccatggattcggctctcatttctatggagggatctctcctgtagcaccagggattctttatctccgtatgtatcgtgctctcagagatactgtctccgtatcggaagagattctctctctgctgatacattcggtggaggatatagcccagaacaggctctcaaaggagaagctggaaaagctgaagacagtgaagaacggatcaagatacggccaaagttctctagcaacagcaatgactcaagtgaagcttgctgcttcgctgagtgcctcgttggtatggctaaccggtggcttaggtgtggtccacgttctcatcaaagaaacgatcccatcttggtttctatcaactgacaaatcagaccgagaacaaggaccatcggatctagttgcagagctaagaggtcatgcccttgcctactttgtggtcctatgtggagcacttacttggggcgtggattcaagatcatctgcatccaaacgccgccgccaagcaatcttgggaagccacttggagttcatcgcgagtgccttagatgggaaaatatcagtgggatgtgaaacagcaacttggcgaacatatatttccggtttggtgagtctaatggtgagctgtttgccgctctgggtgacagagatcgatacagaagtgttaaagagtctaagcaatggactaagaaaatggggaaaagacgagctagcgattgtattactctccttgggaggtctcaaaacaatggattacgcagctgatttcatcattcatttgcgttcatag

SEQ ID NO: 4 - protein, 1309 aa, Petunia x hybrida
MVVPGRRTVWDCVIELTKMAQENCVDPRLWASQLSSNLKFFAVELPSTELAEVIVSYICWDNNVPIVWKFLERAMALKLVSPLVVLALLADRVVPTRSTQQAAYRIYLELLKRNMFTIKDHISGPHYQKVMISVSNILRLSELFDLDTSKPGVLLVEFVFKMVSQLLDAALSDEGLLELSQDSSSQWLVKSQDMEIDAPERYNEKTGSLEKLQSLNTIMAIELIAEFLRNTVIARLLYLVSSNRASKWHEFVQKVQLLGENSSALKHSKVLNSGDLLQLISNRRFGYSYDSKVTSARKSNAIVDFGSLSSYAGLCHGASLSSLWLPLDLVFEDAMDGYQVNPTSAIEIITGLAKTLKEINGSTWHDTFLGLWIAALRLVQRERDPIEGPIPRLDTRLCMSLCIVPLVVANLIEEGKYESVMEKLRDDLVTSLQVLGDFPGLLAPPKCVVSAANKAATKAILFLSGGNVGKSCFDVINMKDMPVNCSGNMRHLIVEACIARNILDMSAYSWPGYVNGRINQIPQSLPNEVPCWSSFVKGAPLNAAMVNTLVSVPASSLAELEKLFEVAVKGSDDEKISAATVLCGASLTRGWNIQEHTVEYLTRLLSPPVPADYSRAENHLIGYACMLNVVIVGIGSVDSIQIFSLHGMVPQLACSLMPICEEFGSYTPSVSWTLPSGEAISAYSVFSNAFTLLLKLWRFNHPPIEHGVGDVPTVGSQLTPEHLLSVRNSYLVSSEILDRDRNRKRLSEVARAASCQPVFVDSFPKLKVWYRQHQRCIAATLSGLTHGSPVHQTVEALLNMTFGKVRGSQTLNPVNSGTSSSSGAASEDSNIRPEFPAWDILKAVPYVVDAALTACTHGRLSPRQLATGLKDLADFLPASLATIVSYFSAEVSRGVWKPVFMNGVDWPSPATNLSTVEEYITKILATTGVDIPSLAPGGSSPATLPLPLAAFVSLTITYKIDKASERFLNLAGPALECLAAGCPWPCMPIVASLWTQKAKRWFDFLVFSASRTVFLHNQDAVIQLLRNCFSATLGLNAAPMSNDGGVGALLGHGFGSHFYGGISPVAPGILYLRMYRALRDTVSVSEEILSLLIHSVEDIAQNRLSKEKLEKLKTVKNGSRYGQSSLATAMTQVKLAASLSASLVWLTGGLGVVHVLIKETIPSWFLSTDKSDREQGPSDLVAELRGHALAYFVVLCGALTWGVDSRSSASKRRRQAILGSHLEFIASALDGKISVGCETATWRTYISGLVSLMVSCLPLWVTEIDTEVLKSLSNGLRKWGKDELAIVLLSLGGLKTMDYAADFIIHLRS

SEQ ID NO: 5 - DNA, 3951 bp, Petunia x hybrida
atggaactatctgtgaagtgcaacatttgggacagtgttttggagttaactaaagtggctcaagaaaagggtggtgatccatttgtatgggcaatacaagtatcttcatgtttaagctcagcaggggtgtctttgccttctcttaagttagctgatttcttggtatcttacatttgttgggaaaataatttgcctatagtatggaagttcttggaaaaggctttggttctcaagattgtgtctcccatcattgtttttcctttgctttccagcagggtaatcccaaatcggcagttgaggcctgcagcatttaggctctacatggaactcctcagaagacatatttttactcttaaaatccatgtaaacatgcaaagttatatgaagattatgaatttcttggacaatgtcctcagtttgactgagatatttggcatgcatgctgatgagcatggtgtgctggttgttgaggttatcttttcaattgtttggcaattactcgatgcatcattagacgatgaagggttgctccaacttactccagaaaagaaatctagatggccaactaagcctgacgatatggagatagatggctacattacagatatggagagaaatgaacacaaagagcgactgaagaatttaaacactcttctggcgattgagatcatagggcaatttttacaaaataaagtaactggcaggattctttacttggctcgccaaaacatgcctgtacattggggagcttttgttcagcgtatacaactacttgctggaaattcatcagcattgcaaagttcaaatgttatatcacccgatgcacttcttcatctggcatcagatgctaacaaattaatgtcctggggcaaaacaaattccttgcaagcacgatctctggctacttgtgctggtctttgtttggggagtagccgttcctccctttggcttcctctagatctcttcctggaagatgcaatggatggttcacaagttaatgctacaagcgccattgaaattattactgacttagtcaaatccctacaagcaatcaatgctacaacttggcatgagatgtttttaggactttggatggcagctctacgacttgttcaaagggagagggatccaattgaaggtcctgtgcctcgcctggatactcgcttgtgcatgttgttctctatcataacacttgtcattgctgatcttattgaggaagaggaaagtgaagcatgtgatgaaattaaatccagcataggcaggaacatgcaaaagcaggttgcaggcacacggcatagggatctagtctcttgtttacagaatcttggtgattatcaaagcttgttaattccacctcaggcaatcactactgcagccaaccaggtcgctgctaaagcaatgatgtttagatcaggcatcaataccaacacgtcatactttgagtgcatcaacatgaaggatatgccaaccaactgttctggtaacctgtatcatttgatagttgaggcatgcattgctagaaatctgctagacacttctgcttacttctggccaggctatgtgaatggacgcatgaaccagttacctcacagtatgccgactcaagtgcctggttggtcatcatttattaagggtgccccattaactccagcaatgattactgctttggtttctgctcctgcttcaagcttagcagagcttgagaagatattcgagatggcagtcagaggagcagatgatgagaagatagctgctgctactattctttgtggagcctcactaattcgtggttggaatatccaggaacatactgtcaatttcataacaagactattgtctcctccagtccctactgactattctggtaaagatagccatttgataggctatgctccaatgttaaatgtcttactcgtcggaatagcaccagtggactgtgttcagattttctcccttcatggcatggtcccacaacttgctgcttcattaatgacaatctgtgaggtgtttggctcatgcgttccgaatatctcctggacactggcaacaggagaggacatctcagtgcatgctgtgttttcaaatgcctttgctctacttttaaagctatggagattcaaccatccacctattgaatatcgagtcggagatgtgccccctgtgggatgccaactaactcctgaatacctcctattagtgaggaattcccatcttgtttcttctgaaaacatgctaaaggacccaaatagaaggagacttgcagctgttgcaagttcatcatctcctaatccaatattactggactcatttccaaaattgagagtttggtatagacaacacctggcatgtatagcttcaacattgtctggtcttgtgaatgggactcttgtctgtcaaactgttgatgttttactcaatatgatgttcaaaaagattaacggggggagtcagagtttgatttccataacatccgggagtagtagctcttctggaactggaagtgaggacacttctccaaggcctaaattacctgcttgggatatacttgaagccgtcccttttgtggttgatgctgctctaacagcatgcgccaatggaagactttctcctcgtgaactgtgcacaggacttaaagatttggctgattttcttcctgcatcactggcaaccattgtaagctacttctctgctgaggtcactcgtggtgtctggaaaccagttgttatgaacggcactgattggccgagtcctgccgcaaatctgtccaatgttgagggacagatcaagaaaattctagcagccactggtgtggatgttccaagcctcgctacaggtggcagctctccagcagttcttccactgccattggctgcatttgtcagccttactataacatacaaactcgataaagcctcccaacgttttctcaatctggcaggccccgctctggagtcacttgcagctggctgcccctggccttgcatgccaattgtggcttccttgtggacccaaaaggcaaaacggtggagtgacttccttgttttttctgcatctcgaactgtctttcttaacaacaaccatgcagtcattcagcttctcaaaagttgtttcctttccgctcttggtttgaactcttcctccatatcaagcaatggtggccttggtgcacttcttgggcacggatttggatcacatttctacggtggtatttctccagttgccccaggcattctgtatctacgagtctacagatccatcagagacatcatgttcttgagagaagagattgtttctcttctgatgcaatctataagtgatatagcatatagtgagcttccaagacaaaggttggacaagcttaagatgccaaaaaatggaaagaaatttggtaatgtttcacttgctacaacaatgacaagggtgaaactagctgccttgcttggggcttctttattatggttatctggtggctcagggctggtacagtcattgataaaagagactcttccctcttggttcttatctgttcacagctctaatcaagaaggtggcaagggtgatctagttccaatgctcaaggggtatgcactcgcatactttgcagtactatgcggagctttcgcaagcggtgtggactcattgtccatggcatcaaagcgacgtccaaagattattgggcgccacgtggaattcatagccaatgtcctggatggcaaaatatcacttggttgtgatccagctacctggcatgcatatgtgtccggatttgtgagcctgatggtaggttgtacaccaacttgggtgtttgaagtggatgctgagttgttaaagaggctcagtaaaggcttgagacaatggcatgaggaggacctcgctcttgctttgcttagcattggtggaattggcacaatgggatcagctgcagagttgatcatggagtcttcgtcatag

SEQ ID NO: 6 - protein, 1316 aa, Petunia x hybrida
MELSVKCNIWDSVLELTKVAQEKGGDPFVWAIQVSSCLSSAGVSLPSLKLADFLVSYICWENNLPIVWKFLEKALVLKIVSPIIVFPLLSSRVIPNRQLRPAAFRLYMELLRRHIFTLKIHVNMQSYMKIMNFLDNVLSLTEIFGMHADEHGVLVVEVIFSIVWQLLDASLDDEGLLQLTPEKKSRWPTKPDDMEIDGYITDMERNEHKERLKNLNTLLAIEIIGQFLQNKVTGRILYLARQNMPVHWGAFVQRIQLLAGNSSALQSSNVISPDALLHLASDANKLMSWGKTNSLQARSLATCAGLCLGSSRSSLWLPLDLFLEDAMDGSQVNATSAIEIITDLVKSLQAINATTWHEMFLGLWMAALRLVQRERDPIEGPVPRLDTRLCMLFSIITLVIADLIEEEESEACDEIKSSIGRNMQKQVAGTRHRDLVSCLQNLGDYQSLLIPPQAITTAANQVAAKAMMFRSGINTNTSYFECINMKDMPTNCSGNLYHLIVEACIARNLLDTSAYFWPGYVNGRMNQLPHSMPTQVPGWSSFIKGAPLTPAMITALVSAPASSLAELEKIFEMAVRGADDEKIAAATILCGASLIRGWNIQEHTVNFITRLLSPPVPTDYSGKDSHLIGYAPMLNVLLVGIAPVDCVQIFSLHGMVPQLAASLMTICEVFGSCVPNISWTLATGEDISVHAVFSNAFALLLKLWRFNHPPIEYRVGDVPPVGCQLTPEYLLLVRNSHLVSSENMLKDPNRRRLAAVASSSSPNPILLDSFPKLRVWYRQHLACIASTLSGLVNGTLVCQTVDVLLNMMFKKINGGSQSLISITSGSSSSSGTGSEDTSPRPKLPAWDILEAVPFVVDAALTACANGRLSPRELCTGLKDLADFLPASLATIVSYFSAEVTRGVWKPVVMNGTDWPSPAANLSNVEGQIKKILAATGVDVPSLATGGSSPAVLPLPLAAFVSLTITYKLDKASQRFLNLAGPALESLAAGCPWPCMPIVASLWTQKAKRWSDFLVFSASRTVFLNNNHAVIQLLKSCFLSALGLNSSSISSNGGLGALLGHGFGSHFYGGISPVAPGILYLRVYRSIRDIMFLREEIVSLLMQSISDIAYSELPRQRLDKLKMPKNGKKFGNVSLATTMTRVKLAALLGASLLWLSGGSGLVQSLIKETLPSWFLSVHSSNQEGGKGDLVPMLKGYALAYFAVLCGAFASGVDSLSMASKRRPKIIGRHVEFIANVLDGKISLGCDPATWHAYVSGFVSLMVGCTPTWVFEVDAELLKRLSKGLRQWHEEDLALALLSIGGIGTMGSAAELIMESSS

SEQ ID NO: 7 - DNA, 4005 bp, Petunia x hybrida
atggaaacaccaacattacacagcaaccaatgggacaaagtaatagagctaaccaaattgggccaacaaagaaacagtgatcctttaatatgggccatgcaaatttcttccattctcaattcagccactatatctatgccatcaacacaattggctaagcttctagtaaatcatatatgttggtccaataacattcctattgcttggaagtttttagaaaaagctttaactctcagacttgtccctcctttgttccttttatctcttctctccaccagagtggttcctagtcgaaggaggtatcccgcagcgtacaggctgtatatggaacttttgaaaagatatgctttttcattaccatctttagtaaatggtcctaattatcaaatgattatggaatcaataaatgatacgcttcatctttccgagatatttgaacatcagggatctgaaagtggtatacatgtggttgaatatgttttcgcggttgtatggcagttactcgatgcctcacttgatgatgagggattgctggaactgactgctgaaaagaagtctaggttgccagttacagctcaagaaatggaaattagcaatcatgatggctttgctgggaaaagattagaacatcctggattatctagaacgaacactatactggctattgaaataattggtcaattttttcaggacacattaacttccatgattctttacttggcacgtaagaacatgcccaaacattgggagtctttcatgcagcacttacaacttctagcgtcaaactcgtcagctcttagaaactctaaggatatctctccggaggctttggtgctgttaatatctaacaatggtggagtcctctccaggaaatgtaaaacaagctcacggcaattccttcgtgctgttatggcttctgcgccacttgcatcttctgctcgtcagtgtgatggtgctagtccatcagttatctggcttcccattgatctctttctagaagataccatggacggatcacaagtggcggctacaagtgctgctgacacacttaccggtctggtgaaggctctgcacgcagttaaccgtacaacgtggcaggacacattttttggtttatggatttcagccctaaggcttgttgatagagaaagggattcaattgagggaccagtacctcgtcttgatacctgcttaagcttattgttatctattacaccgctagcaattaccaacataattaaagaggaggaaaatgatagcagctctagagatcaaagggaagagtccacagcaaaacgtcatcaggccttggtttctagcttacagcaactgcatgattatgatggcttgttgaccccaccactgcctgctattccgttggcaaatcaagctgctttgaaagcaatgatgttcctgtcaggtctaagtgtggggagcgggtattttgatgggatgaggctgaatgacatgcctgtcaattgtgccggaaacttgtggcaccttattgttgaggcttgcatcgctagaaacattttggatacatctgcttatttgtggcctggatatgtaaaaggtcaatttaatcaagtgcctcatagcatgtcaggcccattgcctggctggtcgtcattgatgaaggggtctctcctaactcctccaatggccagtgcattggtttcaacacccgcctcaagcttagcagaaatacagaaaatatatgagatagctgtcaatggtccagatgaagacaagatttctgctgctactattctctgtggggcctctcttgctcgtggttggaatatacaggaacatacggttctattcatcactgagttgctttcacctagtgttccttctgattgtagtggaactgaaagccatttgataggctatgctccatttctgaatgttctcctcgttggcatatcatctattgattgtatcaagattttttctttgcatggattggttccacagcttgctggtgcattgatgccaatttgtgaagctttcggttcttgtgcacccaatgtgtcatggactctgatgtcggaagaaattacttcacatgccattttctcgaatgcattcacacttctgctgaagttgtggaggtttgatcagccacctcttgagcatgtcacaagagatgctactgtgggatcccatctaactcctgaataccttttgctggttcgcaactcccagttggcgtgctctgacgatttgctgatagatcaaagcaaaagcaagaaattgtctgcagctccaagtccatcaccgggagaactgatatttatggattcatttccgaaactaaagcattggtaccggcaaaatcaagcatgtattgcttcacctctctcaggtcttgtccctggaacacctgttcatcagatagtcgaagcactgctaaacttcatgttcagaaaaataagtactggtggtcaatcccttacacctgtgacttcaggtggtagcaactcatctggatctggaaatgaaaatatatctcctcatcttaagctgccagcatgggatattttagaagctgttccgtttgtgcttgatgctgctctcacagcctatgctcatgggagattgtcgccacgtgaactagccacaggtcttaaggatctagctgactttcttcctgcgtctttggcaacaattgcaagttgcttttcagctgaagtgacacggggtatctggaaacctgcttctatgaatggaactgattggccaagtccagctgcaaacttagcaacagtcgaacaacaaataaagaaaatcctagctgccactggtgttgatgtcccaagtctctctgtaggaagttctccagctatccttcctttgcccctggcagcctttgtgagcctcaccattacatataaacttgatagaggtactgagcgctttctgaacttgatgggcccagcactgagtaacctggctacgagttgtccttggccatgtatgccagttgtagctgcgctatgggctcaaaaggttcggcgttggagtgactttcttgttttctctgcatcccggactgtgttccaccatagcagggatgcagtggttcagctgcttcgggtctgctttacggctacactaggtctaggtacatcttccctcgaaagtaatggtggtgttggttcacttcttggtcatggatttggctcacatttttctggtggaatatctcctgttgcccctggtatactctacttgcgcgttcatagagctgtcagaaatgtcatgtttatggcagaagagatcgtcgcccttctgatgcattttgtcagagatattgcggatagtggattccctgctatggaattggagaaactcaggaaacctagaaatgacataaggtctctctcttgccagaaaacgtgtagtgcattcccagcatgttgtcaggtctctcttgctggagcaatgacccgtgtcaagcttgcagcttcattgggtgcttcactggtttggattacaggtggtttaagtttggtccagtctttgctaaaagaaacattgccatcttggtttatatcagcacatgggtctgagcccaacggtgggatctcagaagggatggttgcaaggttgaggggttatgcccttgcatactttgcattgctgtctggagcatttgcttggggtgtggattcattgtcacccacatccaaatggcggccaagtattcttggagcacacctggaatttcttgtgagtgcactagatgggaaaatatcacttggttgtaacaaggctacatggagagcgtatgtttcaggattcataagcttgatggtgggatgcacaccaagctggcttttggaggtggatgttgaggtcttgaagaggctgagccatggattgaaacggtggaacgaggaagtattagctttggccctattggaaactagtggtgttggtgctatgggcactgctgcagaaatgattatgggaggtggtttgaacttttttagctga

SEQ ID NO: 8 - protein, 1334 aa, Petunia x hybrida
METPTLHSNQWDKVIELTKLGQQRNSDPLIWAMQISSILNSATISMPSTQLAKLLVNHICWSNNIPIAWKFLEKALTLRLVPPLFLLSLLSTRVVPSRRRYPAAYRLYMELLKRYAFSLPSLVNGPNYQMIMESINDTLHLSEIFEHQGSESGIHVVEYVFAVVWQLLDASLDDEGLLELTAEKKSRLPVTAQEMEISNHDGFAGKRLEHPGLSRTNTILAIEIIGQFFQDTLTSMILYLARKNMPKHWESFMQHLQLLASNSSALRNSKDISPEALVLLISNNGGVLSRKCKTSSRQFLRAVMASAPLASSARQCDGASPSVIWLPIDLFLEDTMDGSQVAATSAADTLTGLVKALHAVNRTTWQDTFFGLWISALRLVDRERDSIEGPVPRLDTCLSLLLSITPLAITNIIKEEENDSSSRDQREESTAKRHQALVSSLQQLHDYDGLLTPPLPAIPLANQAALKAMMFLSGLSVGSGYFDGMRLNDMPVNCAGNLWHLIVEACIARNILDTSAYLWPGYVKGQFNQVPHSMSGPLPGWSSLMKGSLLTPPMASALVSTPASSLAEIQKIYEIAVNGPDEDKISAATILCGASLARGWNIQEHTVLFITELLSPSVPSDCSGTESHLIGYAPFLNVLLVGISSIDCIKIFSLHGLVPQLAGALMPICEAFGSCAPNVSWTLMSEEITSHAIFSNAFTLLLKLWRFDQPPLEHVTRDATVGSHLTPEYLLLVRNSQLACSDDLLIDQSKSKKLSAAPSPSPGELIFMDSFPKLKHWYRQNQACIASPLSGLVPGTPVHQIVEALLNFMFRKISTGGQSLTPVTSGGSNSSGSGNENISPHLKLPAWDILEAVPFVLDAALTAYAHGRLSPRELATGLKDLADFLPASLATIASCFSAEVTRGIWKPASMNGTDWPSPAANLATVEQQIKKILAATGVDVPSLSVGSSPAILPLPLAAFVSLTITYKLDRGTERFLNLMGPALSNLATSCPWPCMPVVAALWAQKVRRWSDFLVFSASRTVFHHSRDAVVQLLRVCFTATLGLGTSSLESNGGVGSLLGHGFGSHFSGGISPVAPGILYLRVHRAVRNVMFMAEEIVALLMHFVRDIADSGFPAMELEKLRKPRNDIRSLSCQKTCSAFPACCQVSLAGAMTRVKLAASLGASLVWITGGLSLVQSLLKETLPSWFISAHGSEPNGGISEGMVARLRGYALAYFALLSGAFAWGVDSLSPTSKWRPSILGAHLEFLVSALDGKISLGCNKATWRAYVSGFISLMVGCTPSWLLEVDVEVLKRLSHGLKRWNEEVLALALLETSGVGAMGTAAEMIMGGGLNFFS

SEQ ID NO: 9 - DNA, 3984 bp, Petunia x hybrida
atggccaccgtacaatcaagcttatacgacggagtaacagaactaatcaagtcgggtcaggcaaaaggaaccgacccattaagttgggccatgcaactttcttcaaaccttaattcttctggacattctatgccttccattgaagttgccaatcttgttgttaatcatatttgttgggctaataatgttcctattacttggaagtttctagaaaaagctttagctcttcgtattgttcctcctatcgtgattcctgctcgaaggagatacccagtggcgtacaggctatatatggaacttctaaaaagatatgctttctctttgccatctctgattaatggtccaaattaccaaaagatcatggaatcaataaatgacacattacatctttcccagatatttggacttcagggatctgaaagtggtttaattgtggttgaatttatttttacggtcgtatggaagttgcttgatgcgtcccttgatgatgagggattgctggagctgactgctgaaaagaagtctaggtggcctattacatcacaagacatgggactgaatagtcatgatggttttgctggggggagaacagaaaagcatgaagaattatgtaaaatgaacactgttatggctattgaaataattggtgaatttttccgtgataaagtgacttcggcgatcctttttttggcacgcacgaacatgcccacacattgggagtgttttacccagaatttaagacttctcgtgtcaaactcatcagctttaagaaactctaagaatatttctccggaggctttggtgcagttaacatccaataattgtgtagtcctctctagaaaatgcaaaacaagctcacagaaaaggtcccatgctgtaatggcttctggttcacttgcatcttctgcaaatcactgtcatggtgctaattcatcggtgctttggcttcccatcgacctctttctagaagataccatggacgggtcacaggcagcggctacaagtgctgctgaaacactcactggtctagtgaaggctctgcaggccatcaactctaccacctggcaggacacttttctaggattgtgggttgcggccctaaggcttgtcaatagagaaagggattcaatcgagggaccagtacctcgcctagatacttgcttatgcttgttgttgtcgattacaccactagcaatagtgaatattattgaagaggaggaaaggaactgcggctcgaccaatcaaaggaaggagtcctcaggaaaacgtcgccaagacttggtatatagccttcagcaacttgatgattatgaaggcttgttgaccccaccgctaccttctactccattggcgaatcaggctgcggcaaaggcaatgatgttcctttcaggtttaagcgtcgggagtggctattttgaaggaatgagcttgaatgacatgcctgtcaattgcgctggaaacctgcggcacttgattgtcgaagcttgcatttccagaaacattttggacacgtctgcttatttgtggcccggatatgtaaaaggtaaatgcaatcaagtgccgcgtagtgtctcgggccaaacgcctggctggtcatcactgatgaaggggtctcctttagctcctccgttggtcagttccttggtatcaactccagcatcaagtttagcagaaatagagaaaatatatgagtttgccgtcaacggctcggatgatgacaagatttctgctgctactattctctgtggcgcctctcttgctcgtggttggaatgtacaggaacatacggtactgttcatcaccaggttgctttcacctcccgttcctgctaattattctggaactgaaagtcatttagttggctacgctccgtttctgaacgttcttctcatcggaatgtcatctattgattgtgttcagattttttctttgcatggattggttccacagcttgctggtgcattgatgcctatttgtgaagcctttgggtcatgtgcacccaatgtgacatggactgtaatgtctgaagaaatttcttcacatgctgttttctcaaatgcatttactcttctgttgcggttgtggaggtttgatcagccacctcttgaaaatgaattagatgttccagtgggatcccgtctaactcctgaataccttttgctggtgcgcaactcccagttagcatcctctgatgatctgaataaagatcaaagcaaaatcaagcggctggctaaactttcaagtccattatctggagaacccatatatgttgattcttttccgaaattaaaactttggtacagacaaaatcaagcatgcattgcttcaccactctctggtcttgtccctggaactcctgttcatcagatcgttgaagcactattgaacttcatgttcagaaaaataaatagaactggtcagtcactgacgccaactacttctggaagcagtagctcatctgggcctggaaacgaagacgtatctctccaccttaagctgcctgcatgggacattctagaagctgctccttttgtgcttgatgccgctctcactgcatgtggtcatggtagattgtcaccgcgtgaactagccacaggtcttaaggatctggctgactttctcccagcgtctttggcaacaatcgtaagttacttttcagctgaagtaacacggggtatctggaagcttgcttctatgaatggaactgattggccaagcccagctgcaaatttagcaacagtggagcaacagataaagaaaattttagctgcaactggtgtgaatgtcccaagtctcaccgtaggtggaaattctccatcaacccttccattgcccctggcagcccttgtgagccttaccattacatacaaacttgatagatccactgaacgctttctgaacatgatgggccctgccttgagtaacttggctacaggttgtccttggccctgcatgcctgtcatggctgcgctatgggcccagaaggtgaagcgctggagtgactttctggttttctcttcatcccggactgtgttccaccacaatactgatgcagtggttcaactgcttagagtgtgctttgcagctacactaggtctatctacatcttctatcacaagcaatggcggtgttggtgcacttcttggtcacggttttggttctcatttttctggtggtatctccccggttgcccctggtatactctacttgcgcgtgcatagagctgtccgaaatgtcatgtttatgacagaggaagttgtctcccttttgatgcattttgtcagagaaattgcaagtagtggattaccttctgagaaattggagaaactcaagaaaagtaaatgtggtatgaggtatggtcaggtttctcttgctgcggcaatgagtcgtgtaaagcttgcagcttcactgggtgcttcattagtttggatcacaggtggtgaaggtttggttcaatcattgattagagaaaccttgccatcatggtttatatcagcacatgggtcggagcaagctggtgggatgtcaggaggcttagttgcaatgcttaggggttatgccctcgcttactttgcagtgctgtctggaacatttacatggggggtagattcatcatcagcttcatccaagcggcgggcaagtattcttgagtcacacttggaattccttgcaagtgctctagatgggaaaatatccctcggttgtgacagggcaacctggagagcttatgtttcagcgttcctgcgcttgatggtgcggtgcacacctagctggatgctggaggtggatgtagagattttgaagaggctaagtatgggattgaaacagtggaatgaggaggagttagcattggctcttttgagtagtggtggtattagtgcaatgggggctgctgctgaaatgattatagaaggagtaccccatatagttgacaaagaaaccaaattgttgaaacctgctcatttactgggaactgtgtattag

SEQ ID NO: 10 - protein, 1327 aa, Petunia x hybrida
MATVQSSLYDGVTELIKSGQAKGTDPLSWAMQLSSNLNSSGHSMPSIEVANLVVNHICWANNVPITWKFLEKALALRIVPPIVIPARRRYPVAYRLYMELLKRYAFSLPSLINGPNYQKIMESINDTLHLSQIFGLQGSESGLIVVEFIFTVVWKLLDASLDDEGLLELTAEKKSRWPITSQDMGLNSHDGFAGGRTEKHEELCKMNTVMAIEIIGEFFRDKVTSAILFLARTNMPTHWECFTQNLRLLVSNSSALRNSKNISPEALVQLTSNNCVVLSRKCKTSSQKRSHAVMASGSLASSANHCHGANSSVLWLPIDLFLEDTMDGSQAAATSAAETLTGLVKALQAINSTTWQDTFLGLWVAALRLVNRERDSIEGPVPRLDTCLCLLLSITPLAIVNIIEEEERNCGSTNQRKESSGKRRQDLVYSLQQLDDYEGLLTPPLPSTPLANQAAAKAMMFLSGLSVGSGYFEGMSLNDMPVNCAGNLRHLIVEACISRNILDTSAYLWPGYVKGKCNQVPRSVSGQTPGWSSLMKGSPLAPPLVSSLVSTPASSLAEIEKIYEFAVNGSDDDKISAATILCGASLARGWNVQEHTVLFITRLLSPPVPANYSGTESHLVGYAPFLNVLLIGMSSIDCVQIFSLHGLVPQLAGALMPICEAFGSCAPNVTWTVMSEEISSHAVFSNAFTLLLRLWRFDQPPLENELDVPVGSRLTPEYLLLVRNSQLASSDDLNKDQSKIKRLAKLSSPLSGEPIYVDSFPKLKLWYRQNQACIASPLSGLVPGTPVHQIVEALLNFMFRKINRTGQSLTPTTSGSSSSSGPGNEDVSLHLKLPAWDILEAAPFVLDAALTACGHGRLSPRELATGLKDLADFLPASLATIVSYFSAEVTRGIWKLASMNGTDWPSPAANLATVEQQIKKILAATGVNVPSLTVGGNSPSTLPLPLAALVSLTITYKLDRSTERFLNMMGPALSNLATGCPWPCMPVMAALWAQKVKRWSDFLVFSSSRTVFHHNTDAVVQLLRVCFAATLGLSTSSITSNGGVGALLGHGFGSHFSGGISPVAPGILYLRVHRAVRNVMFMTEEVVSLLMHFVREIASSGLPSEKLEKLKKSKCGMRYGQVSLAAAMSRVKLAASLGASLVWITGGEGLVQSLIRETLPSWFISAHGSEQAGGMSGGLVAMLRGYALAYFAVLSGTFTWGVDSSSASSKRRASILESHLEFLASALDGKISLGCDRATWRAYVSAFLRLMVRCTPSWMLEVDVEILKRLSMGLKQWNEEELALALLSSGGISAMGAAAEMIIEGVPHIVDKETKLLKPAHLLGTVY